(19) United States
(12) Patent Application Publication
(10) Pub. No.: US 2026/0174443 A1
(43) Pub. Date: Jun. 25, 2026

(54) PATIENT-MATCHED AND SURGEON-SPECIFIC SINGLE USE SURGICAL INSTRUMENTS SYSTEM

(71) Applicant: Mahmoud Alm El Din HAFEZ, Giza (EG)

(72) Inventors: Mahmoud Alm El Din HAFEZ, Giza (EG); Ahmed Abd Almoghny Salem, Giza (EG); Ahmed Aboulfath Hassan Ali, Cairo (EG)

(21) Appl. No.: 19/132,590

(22) PCT Filed: Nov. 24, 2022
(86) PCT No.: PCT/EG2022/000033

§ 371 Date: May 23, 2025

(51) Int. Cl. A61B 17/15; A61B 17/56; A61B 34/10; A61F 2/46; B33Y 80/00; G16H 20/40

(52) U.S. Cl. CPC A61B 17/155; A61B 34/10; A61F 2/461; B33Y 80/00; G16H 20/40; A61B 2017/568; A61B 2034/108

(57) ABSTRACT

A system of patient-matched and surgeon-specific single use surgical instruments designed for five different applications for knee replacement surgeries; primary total knee replacement, custom-made total knee replacement, revision total knee replacement, hinged total knee replacement and partial knee replacement. The system has patient-matched & surgeon-specific instruments are designed & prepared for each case based on patient's need and surgeon's demand. The system contains patient-matched set of blocks and guides, “On-Demand” surgeon-specific Instruments sets, conventional instruments sets, surgical planning software, 3D printing Software, 3D printers and sterilization unit. All the system components are manufactured by using 3D printing technologies.

### TECHNICAL FIELD

A system of patient-matched and surgeon-specific single use surgical instruments designed for five different applications for knee replacement surgeries; primary total knee replacement, custom-made total knee replacement (primary-revision-hinged), revision total knee replacement, hinged total knee replacement and partial knee replacement. The system has patient-matched & surgeon-specific instruments are designed & prepared for each case based on patient's need and surgeon's demand.

### BACKGROUND OF THE SUBJECT

Prostheses and implants are commonly used in bone and joint surgery. The surgical operations usually require instruments and tools to help the surgeon to implant the prosthesis. Major operations such as joint replacement, spinal surgery and bone tumour surgery are technically demanding. The results of these major surgical operations are dependent on the type of implants and surgical techniques including instruments and tools.

One of the examples for bone and joint surgery is knee replacement. It involves the implantation of the knee prosthesis after sizing the implant and machining (drilling, cutting and/or milling) the joint surfaces to match the internal geometry of the prosthesis. The surgery for knee replacement involves more than 50 surgical steps and usually requires more than 300 pieces of conventional instruments and surgical guides. The aim of knee replacement surgery is to achieve long-term implant survival and successful functional outcome with cost effectiveness and minimal complications. Technical errors can have detrimental effects on function and survival.

### CURRENT ART

Computer-assisted robotics, navigation and patient specific templates (PST) technology have proved to be more accurate than conventional instruments, but they have not gained popularity due to several limitations such as complexity of use, high cost and inability to completely replace conventional instruments. PST has been reported to eliminate the use of intramedullary rods of the conventional techniques with their consequences of potential blood loss, fat embolism, inaccuracy and infection.

The concept of patient-specific templates (PST) has already been exploited by all major implant manufacturers. It has been clinically applied on many joints such as knee, hip, shoulder and ankle. The preparation is not surgeon-based, and it takes long duration for confirming the preoperative planning and fabricating the templates as the company outsources most of the steps of PST process such as planning of surgery and designing of PST. Following template fabrication, the templates return to the company for packing and sterilization. Finally, the company sends the templates to the hospital, so that the surgeon can use during surgery. The process does not only involve outsourcing the steps, but it also involves more than one country. Planning is routinely done by technicians and not by surgeons as both usually work at different locations. The process of PST takes about 4-6 weeks from the time of acquiring the imaging (MRI or CT) until the templates are delivered to the hospital. This may carry the risk of anatomic changes to the knee as a result of daily activities or any abnormal loading during this long delay, resulting in intraoperative malpositioning of the templates and subsequently implant malalignment.

This complex process and logistics limit the availability of PST in developing countries where implant companies are not widely distributed. The surgeons usually need longer time for communication with implant manufacturers to obtain the custom cutting guides and the desired implant. There is an additional high cost for the process of PST that is not proven to be cost effective.

The expensive industrial 3D printing machines that are used to manufacture PST have an average cost of $500,000. But currently the availability and affordability (<$500) of desktop 3D printers make it possible to replace the industrial scale machines.

### REFERENCES

1. Hafez M A, Chelule K, Seedhom B B, Sherman K P. Computer-assisted total knee arthroplasty using patient-specific templating. Clinical Orthopaedic and Related Research. 2006; 444:184-192
2. Patient specific instruments and related methods for joint replacement. U.S. 61/641,851.
3. Device and method for fitting an artificial knee joint using universal electronic templates which can be adapted to all artificial joints. U.S. Ser. No. 10/849,636B2.

### DESCRIPTION OF INVENTION

A system of patient-matched and surgeon-specific single use surgical instruments designed for five different applications for knee replacement surgeries; primary total knee replacement, custom-made total knee replacement (primary-revision-hinged), revision total knee replacement, hinged total knee replacement and partial knee replacement. The system has patient-matched & surgeon-specific instruments are designed & prepared for each case based on patient's need and surgeon's demand.

The system contains patient-matched set of blocks and guides, “On-Demand” surgeon-specific Instruments sets, conventional instruments sets, surgical planning software, 3D printing Software, 3D printers and sterilization unit. These components are not connected either physically or electrically.

Surgical planning software allows he surgeon to plan and predict all the surgical steps visually on the screen based on a specific medical rule including the mechanical axis, anatomical axis, rang of motion, varus/vulgus angel. All of these information helps in detect the correct size of implant without needing to predict the up/down sizes.

The preoperative planning contains three phase which are image processing phase to reconstruct the bone and create a 3D model of the femur and tibia based on the CT-scan images of the patient and segmentation process of its layers, while the second phase is performing the surgical planning including selection of the optimum size, rotation and alignment of the implant, positioning of the implant over the bone, volume of bone removal, and varus/vulgus correction.

Sizing of the femoral and tibial components was done automatically by the system and verified to avoid undue anteroposterior and mediolateral mismatching or any implant overhang in any plane. The planning could reveal information that would not be available to the surgeon during actual surgery, such as posterior tibial overhang or posterior femoral offset.

Alignment (angles and rotation) and bone resection were planned according to the set default for eight standard parameters: femoral coronal alignment, femoral sagittal alignment, femoral rotation, level of distal femoral cutting, tibial coronal alignment, tibial sagittal alignment, tibial rotation and level of tibial cutting. The ideal bone cuts were measured on software with definite length, direction and inclination.

The third phase is seating the patient specific guide over the bone according to the results of the previous phase, and creating the seating surfaces.

The system contains a 3D modeling software that to create the 3D model of the single use instruments based on the conventional instruments of the implant that will be used in the surgery.

After creating the electronic file of the patient specific guides and the single use instruments, they transferred to the 3D printing machines to start the manufacturing process. The printing process performed by filament-based machine (FFF) and/or powder-based machines (SLS).

Finishing and inspection of the printed products will be done under the same roof. Last step in this system is the sterilization process, the products that are manufactured by SLS technology will sterilized in the autoclave, while the products that are manufactured by FFF technology will be sterilized in plasma sterilizer for one cycle with 45 minutes. The material used for the production of patient specific guides and the single use instruments in this system is Nylon-PA2200 Polylactic Acid and/or Stainless-steel which meets certain criteria such as being biocompatible, heat stable to withstand high temperature of sterilization, durable enough not to be damaged by saw blades and relatively inexpensive.

Generally, all of the patient-matched and surgeon-specific single-use instruments in this system are made from medical-grade and 3D-printable materials that are bioabsorbable, absorbable, non-absorbable, biodegradable, non-biodegradable and recyclable material. Wherein, the one or more materials are selected from the material group consisting of stainless-steel, high-density polyamide, polylactic acid, polyethylene, polycarbonate, acrylonitrile-butadiene-styrene, laminates of one or more of these materials, coextruded filament including one or more of these materials, and combinations thereof. One or more material suitable for maintaining sterility of knee replacement surgical instrument contained therein during transportation or storage thereof.

The system is designed to plan, develop, produce, package and sterilize single-use surgical instruments for different applications for knee replacement surgeries. The system is placed inside a healthcare institution or hospital (in-house) or as an independent workshop facility. in this system the custom-made patient-specific single-use instruments solution is an open platform that offers custom-made patient-specific cutting guides and custom-made patient-specific single-use instruments intended to replace conventional reusable instruments, single-use instruments, computer assisted surgery, and robotic-assisted surgery for primary total knee replacement surgery.

This system established inside hospitals to eliminate traditional barriers to accessing the Patient-Specific Single-Use Instruments as they offer distinctive financial and logistical values including:
1. Reduce the average standard cost for one set of patient specific guides for one patient.
2. Reduce the average standard planning timeline for one set of patient specific guides for one patient from 4-7 Days to 45-60 minutes.
3. Reduce the average standard delivery time for one set patient specific guides for one patient from 12-20 Weeks to 24 hours.
4. Being Hospital-Based, they eliminate entire traditional logistical efforts and challenges in connection to the delivery of one set of patient specific guides including shipping, regulatory clearance, and customs clearance.

Producing the patient-matched and surgeon-specific single-use instruments in this system are based on the 2D images of the knee resulting from CT-scans, MRI, Ultrasound and/or X-ray. Wherein the preoperative planning software could reverse engineering the bones and joints using integrated data from multiple sources instead of CT-scans or MRI.

As the system is containing patient specific guides resulting from the preoperative planning, so that it is allowing faster and less invasive surgical procedure through skipping the need of intramedullary and/or extramedullary alignment rod designed to validate implant positioning during surgical process.

In this system the delivering knee replacement surgical instruments based on surgeon's demand, according to surgeon's surgical philosophy and approach to surgical philosophy, while the system is characterized by inclusion or exclusion of a specific surgical instrument(s) is a surgical decision made only by surgeon.

The health care providers have the ability to produce patient-matched and surgeon-specific single-use instruments for any of the knee implants and fulfils surgical instruments for any surgical applications. The system offers three options for the surgeon to choose most appropriate version that suits patient's specific needs and adopted surgical philosophy; each of offered three options consists of femoral component and tibial component. The three options are:
1—Patient specific completing guides cutting guides with selected single use instruments.
2—Patient specific distal femur cutting guides and patient specific proximal cutting guides with full set of single use instruments.
3—Patient specific pin positioning guides with full set of single use instruments.

The first option is patient specific completing guides cutting using to perform a complete cutting, preparation, and finishing femoral & tibial blocks/guides allowing full surgical access to perform all the joint replacement actions and requirements embedded in bodies of complete cutting, preparation, and finishing femoral & tibial blocks/guides.

The femoral cutting guide in option one is a complete cutting guide consisting of distal femoral cut, anterior femoral cut, posterior femoral cut, anterior chamfer cut, posterior chamfer cut, box cut for patient specific femoral component, valgus angle, external rotation angle, Q angle, distal femoral component positioning lugs, anterior position verification marking, anterior grooves for external alignment verification, design-specific anterior cut angle, saw blade-specific cutting-slot thickness and oblique fixation holes.

While the tibial cutting guide in option one is a complete cutting guide consisting of proximal tibia cut, proximal tibia cut posterior slope angle, tibia preparation drill(s), tibia preparation keel/punch, anterior position verification marking, anterior grooves for external alignment verification, saw blade-specific slot thickness and oblique fixation holes.

The second option is patient specific distal femur cutting guides and patient specific proximal cutting guides with full set of single use instruments. This option is an initial cutting blocks/guides offering guides for preparation and finishing of femoral and tibial aspects with features embedded in bodies of initial cutting components.

The femoral cutting guide in option two is patient specific distal femur guide that are consisting of distal femoral cut, valgus angle, external rotation angle, Q angle, 4in1 cutting block marking, distal femoral component positioning lugs marking, anterior position verification marking, anterior grooves for external alignment verification, saw blade-specific cutting-slot thickness and oblique fixation holes. While the tibial cutting guide is a patient specific proximal tibia cutting that are consisting of proximal tibia cut, proximal tibia cut posterior slope angle, anterior position verification marking, anterior grooves for external alignment verification, saw blade-specific slot thickness and oblique fixation holes.

The third option is patient specific pin-positioning blocks/guides offering guides for cutting, preparation and finishing of femoral and tibial aspects with features embedded in bodies of pin-positioning blocks/guides. The femoral guide in this option consisting of distal femoral cut marking, valgus angle, external rotation angle, Q angle, 4in1 cutting block marking, distal femoral component positioning lugs marking, anterior position verification marking, anterior grooves for external alignment verification, saw blade-specific cutting-slot thickness and oblique fixation holes. While, the tibial guide consisting of proximal tibia cut marking, proximal tibia cut posterior slope angle, anterior position verification marking, anterior grooves for external alignment verification, saw blade-specific slot thickness and oblique fixation holes.

All of the patient specific guides in the three options has a marking central slot in the anterior side allows validation of guide position, alignment, and rotation over the bone throughout surgical procedure. To ensuring the positioning and alignment of the implant over the bone, an extra validation feature with be available with all guides. The extra feature is an alignment slot allows the surgeon to fix a multi-sizes alignment plate and rod to ensure the implant alignment along varus/vulgus, internal/external rotation and axial rotation throughout surgical procedure.

Beside the patient specific guide that are mentioned in the above, the system also delivers single-use patient-matched and surgeon-specific conventional instruments based on surgeon's demand with different eleven options to allow the surgeon performing the surgery with several solutions the maximize the accuracy of the surgery and the save the operation room time.

In this system the single-use knee replacement sets of instruments each of these sets is designed for a specific patient based on the surgical needs of such specific patient and surgical philosophy of operating surgeon, the eleven options are that are listed in following:

Option one, is designed for primary total knee replacement, custom-made total knee replacement (primary-revision-hinged), revision total knee replacement, hinged total knee replacement and uni-compartmental/partial knee replacement; consisting of distal femoral cutting block offers open or sloped cut option, minimally invasive or component dimension option, distal-proximal femoral cut translation [on demand (0 mm), (−1/+1 mm), (−/+2 mm) (−/+3 mm) and (−/+4 mm)] options, anterior grooves for external alignment verification, position validation central mark, saw blade-specific slot thickness, and oblique fixation holes.

Option two, is designed for primary total knee replacement, custom-made total knee replacement (primary-revision-hinged), revision total knee replacement and hinged total knee replacement; consisting of distal femoral cutting block offers open or sloped cut option, minimally invasive or component dimension option, distal-proximal femoral cut translation [on demand 0 mm up-to 40 mm in 4 mm increment] options, anterior grooves for external alignment verification, position validation central mark, saw blade-specific slot thickness, and oblique fixation holes.

Option three, is designed for primary total knee replacement, custom-made total knee replacement (primary-revision-hinged), revision total knee replacement and hinged total knee replacement; consisting of 4in1 femoral cutting block offers open or sloped cut option, minimally invasive or component dimension option, anterior-posterior femoral cut translation [on demand (0 mm), (−1/+1 mm), (−/+1.5 mm) (−/+2 mm) and (−/+3 mm)] options, anterior grooves for external alignment verification, position validation central mark, saw blade-specific slot thickness, and oblique fixation holes.

Option four, is designed for primary total knee replacement, custom-made total knee replacement (primary-revision-hinged), revision total knee replacement and hinged total knee replacement; consisting of cutting block for box cut for ps femoral component offers open or sloped cut option, minimally invasive or component dimension option, distal femoral component positioning lugs, anterior grooves for external alignment verification, position validation central mark, saw blade-specific slot thickness, and oblique fixation holes.

Option five, is designed for uni-compartmental/partial knee replacement; consisting of 3in1 femoral cutting block offers open or sloped cut option, minimally invasive or component dimension option, anterior grooves for external alignment verification, saw blade-specific slot thickness, and oblique fixation holes.

Option six, is designed for primary total knee replacement, custom-made total knee replacement (primary-revision-hinged), revision total knee replacement, hinged total knee replacement and uni-compartmental/partial knee replacement; consisting of proximal tibia cutting block offers open or sloped cut option, minimally invasive or component dimension option, distal-proximal tibial cut translation [on demand (0 mm), (−1/+1 mm), (−/+2 mm) (−/+3 mm) and (−/+4 mm)] options, anterior grooves for external alignment verification, position validation central mark, saw blade-specific slot thickness, and oblique fixation holes.

Option seven, is designed for primary total knee replacement, custom-made total knee replacement (primary-revision-hinged), revision total knee replacement and hinged total knee replacement; consisting of proximal tibia cutting block offers open or sloped cut option, minimally invasive or component dimension option, distal-proximal tibial cut translation [on demand 0 mm up-to 40 mm in 4 mm increment] options, anterior grooves for external alignment verification, position validation central mark, saw blade-specific slot thickness, and oblique fixation holes.

Option eight, is designed for primary total knee replacement, custom-made total knee replacement (primary-revision-hinged), revision total knee replacement, hinged total knee replacement and uni-compartmental/partial knee replacement; consisting of trial instruments set offers patient size-specific femoral trial, tibia plate trial, tibia insert trials [surface & multiple shims], flexion/extension gap assessment shims, intra-medullary stem extensions [0/offset tibial & femoral stems], intra-medullary sleeves [tibial & femoral sleeves], intra-medullary cones [tibial & femoral cones], tibia wedges [full, hemi, step & block wedges] and femoral augments [distal, anterior & posterior].

Option nine, is designed for primary total knee replacement, custom-made total knee replacement (primary-revision-hinged), revision total knee replacement, hinged total knee replacement and uni-compartmental/partial knee replacement; consisting of position verification & validation instruments set, allows intra-operative validation to make a double check before performing the final bone resection, offers full limb external alignment plate & rods [plate dimensions, rod diameter & rod length are on surgeon's demand], femoral cut validation c-section tool, tibia bone cut stylus, tibial & femoral bone cuts verification wing, and bone caliber.

Option ten, is designed for primary total knee replacement, custom-made total knee replacement (primary-revision-hinged), revision total knee replacement, hinged total knee replacement and uni-compartmental/partial knee replacement; consisting of bone preparation and finishing set offers femoral lugs drill, tibia preparation drill, tibia keel/punch preparation, sleeves preparation, stem extension preparation, long-stem preparations, cones preparation and other preparation and finishing instruments based on surgeon's demand Option eleven, is designed for primary total knee replacement, custom-made total knee replacement (primary-revision-hinged), revision total knee replacement, hinged total knee replacement and uni-compartmental/partial knee replacement; consisting of impaction & extraction instruments set offers modular impactor/extractor handle, femoral trial & final femoral component positioning impactor/extractor, femoral impactor, tibia base plate impactor, tibia insert impactor.

The system have a balancing block for the purpose of flexion/extension gap balancing, the block contains translation holes with +1, −1, +2, −2 to ensure balancing anterior up of posterior down.

In this system all the single-use patient-matched and surgeon-specific blocks/guides and conventional instruments that are exhibit minimal scratching and no observable debris generation during use. This system operates within specifications of “clean room” to minimize rates of contamination and pollutants such as dust, airborne microbes and aerosol particles during the manufacturing and sterilization process.

### DRAWINGS DESCRIPTION

FIG. 1: 3D view of the femoral pin positioning guide (1) shows the lateral fixation hole (5), medial fixation hole (4), alignment tower base (2), trochlea fixation leg (6) and 4in1 femoral component positioning lugs (3).

FIG. 2: 2D view of the femoral pin positioning guide shows the distal femoral component positioning holes (8) and anterior groove for external alignment verification (7).

FIG. 3: 3D view of the femoral pin positioning guide shows the distal fixation legs (10), superior fixation legs (9) and trochlea fixation leg (6).

FIG. 4: 3D view shows the positioning of the femoral pin positioning guide (1) over the femur bone (11).

FIG. 5: 3D view of the femoral distal cut guide (12) shows the lateral fixation hole (5), medial fixation hole (4), alignment tower base (2), trochlea fixation leg (6), 4in1 femoral component positioning lugs (3) and the distal cutting slot (13).

FIG. 6: 2D view of the femoral distal cut guide shows the distal femoral component positioning holes (8) and the distal cutting slot (13).

FIG. 7: 3D view of the femoral distal cut guide shows the distal fixation legs (10), superior fixation legs (9) and trochlea fixation leg (6).

FIG. 8: 3D view shows the positioning of the femoral distal cut guide (12) over the femur bone (11).

FIG. 9: 2D view of the femoral complete cutting guide shows the anterior cutting slot (17), the posterior cutting slot (14), the anterior chamfer cutting slot (15), the posterior chamfer cutting slot (16), oblique fixation holes (19), the box-cutting slots (18), 4in1 femoral component positioning lugs (3) and the groove for external alignment verification (20).

FIG. 10: 3D view of the femoral complete cutting guide shows the distal cutting slot (13), the box-cutting slots (18) and the groove for external alignment verification (20).

FIG. 11: 3D view shows the positioning of the femoral complete cutting guide (21) over the femur bone (11).

FIG. 12: 3D view of the femoral complete cutting guide shows the superior seating legs (23) and the distal cutting slot extension (22).

FIG. 13: 3D view of the femoral complete cutting guide and femur bone after performing all the femoral cuts, the figure shows the anterior cut (24), the posterior cut (27), the anterior chamfer cut (25), the posterior chamfer cut (28), the distal cut (26) and the superior seating legs (23).

FIG. 14: 3D view of the femoral pin positioning guide shows the distal cut (26) and the superior seating legs (23).

FIG. 15: 2D view of C-Check shape (86)

FIG. 16: 3D view of angle wing (94)

FIG. 18: 3D view of the tibial pin positioning guide shows the proximal tibial component

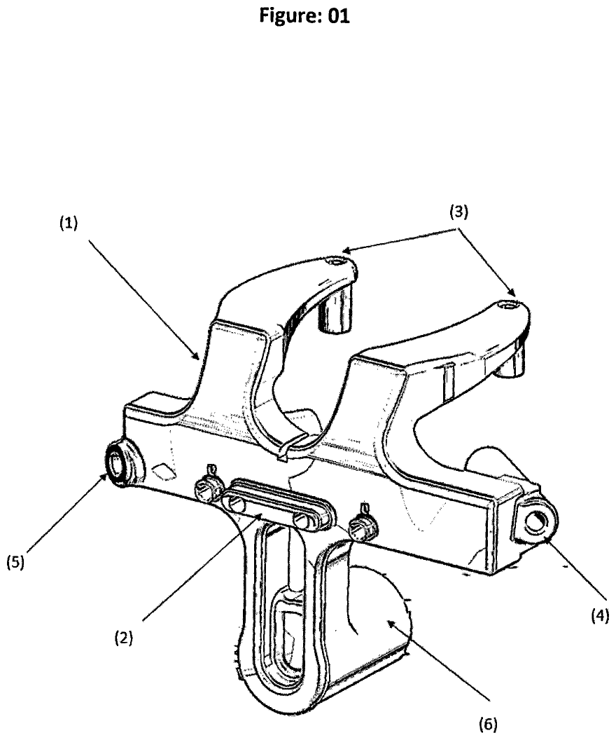

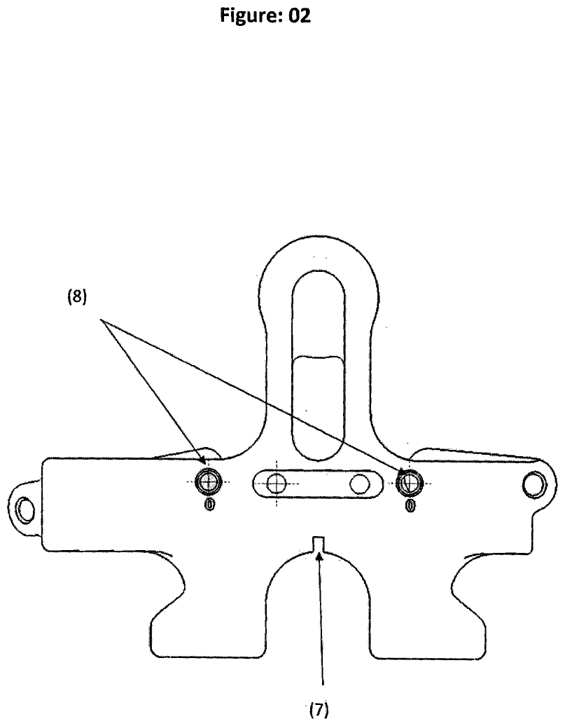

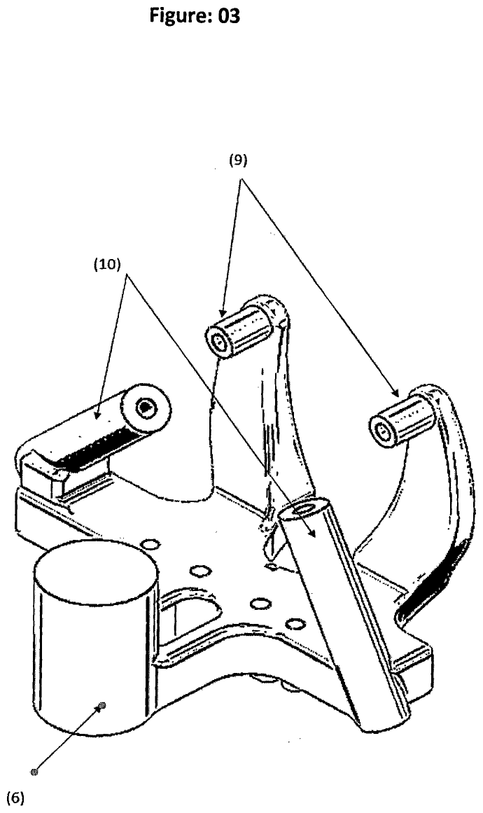

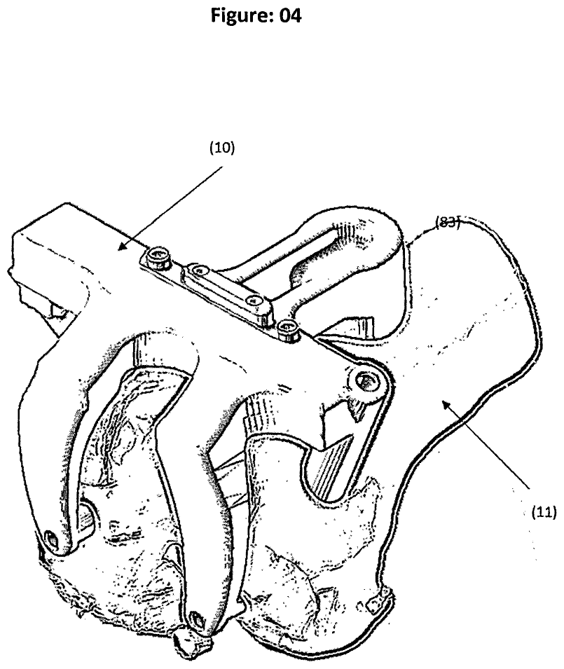

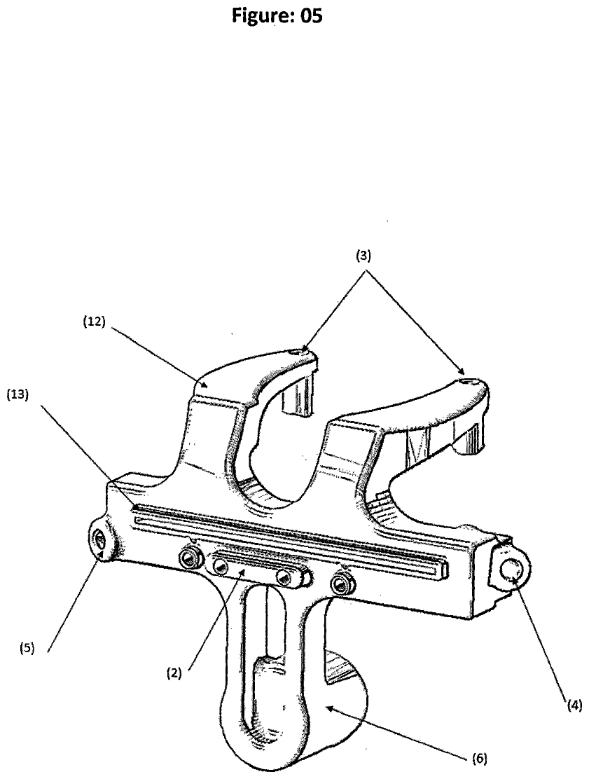

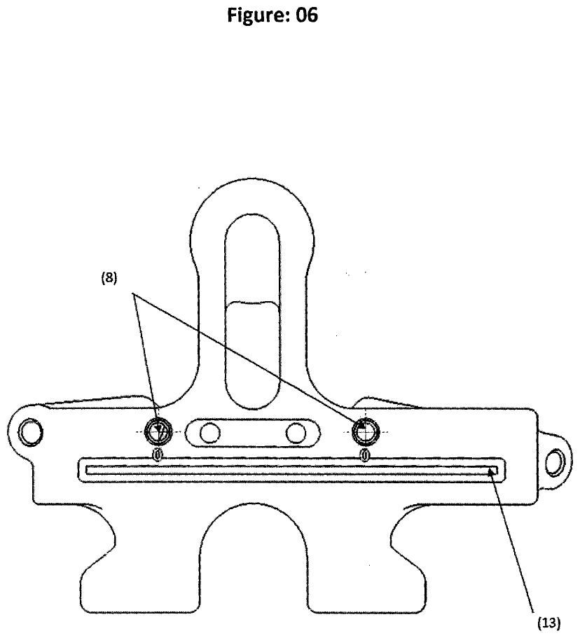

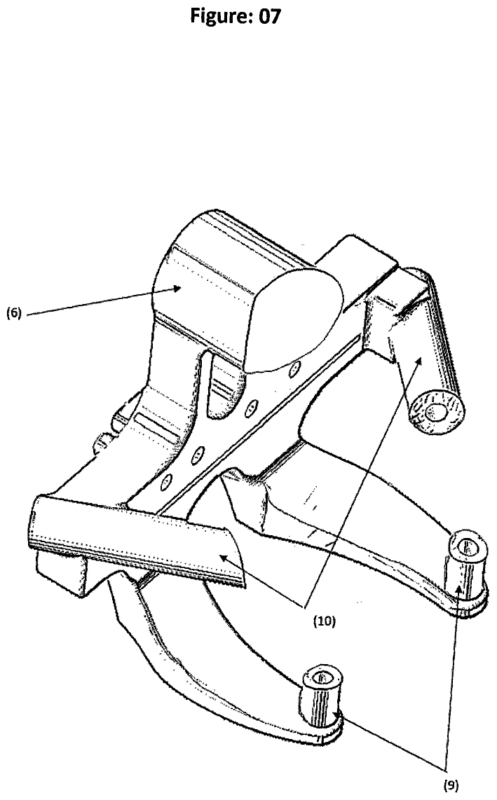

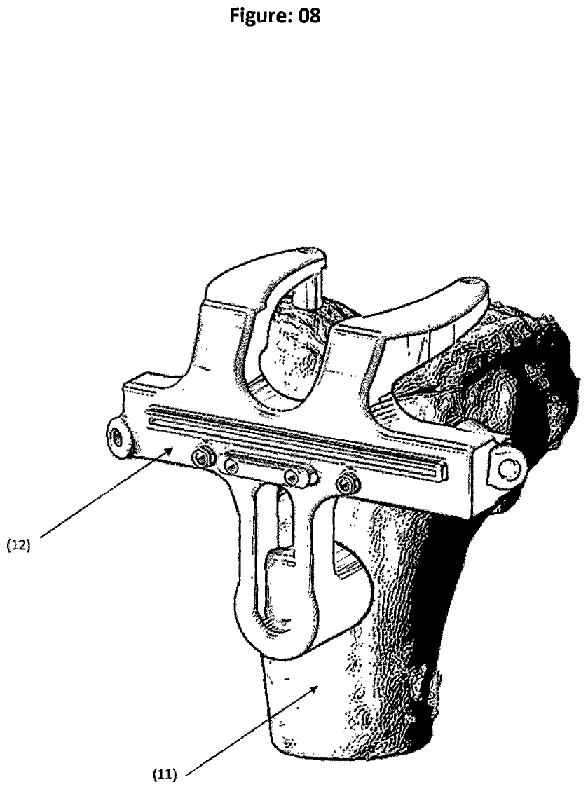

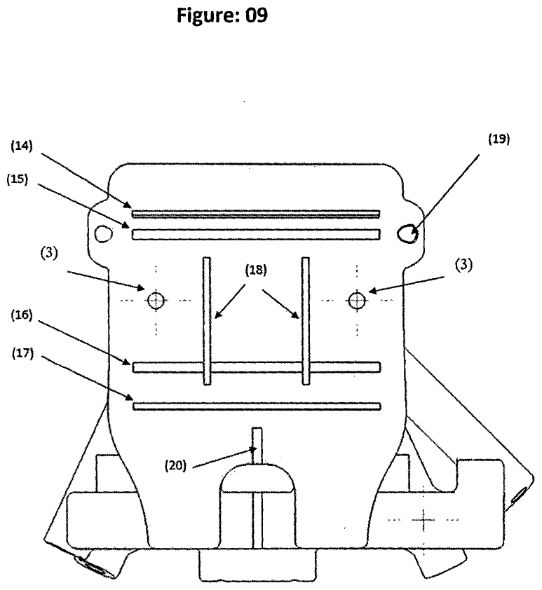

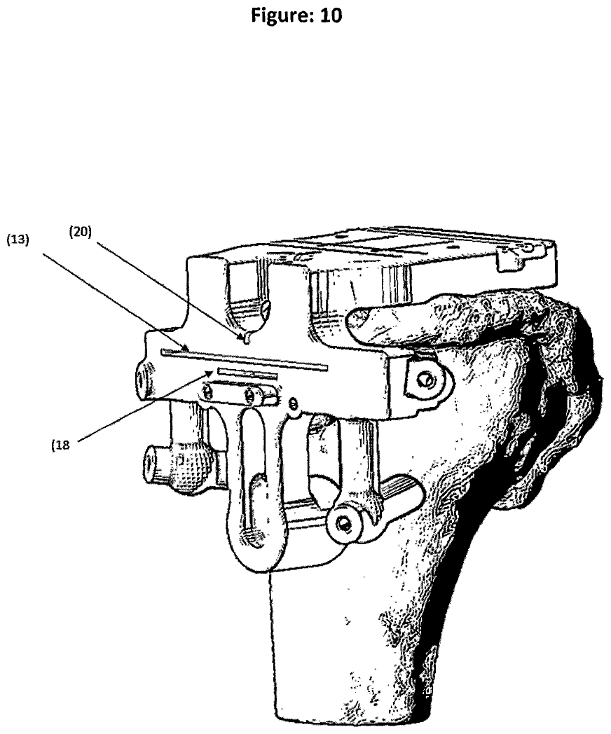

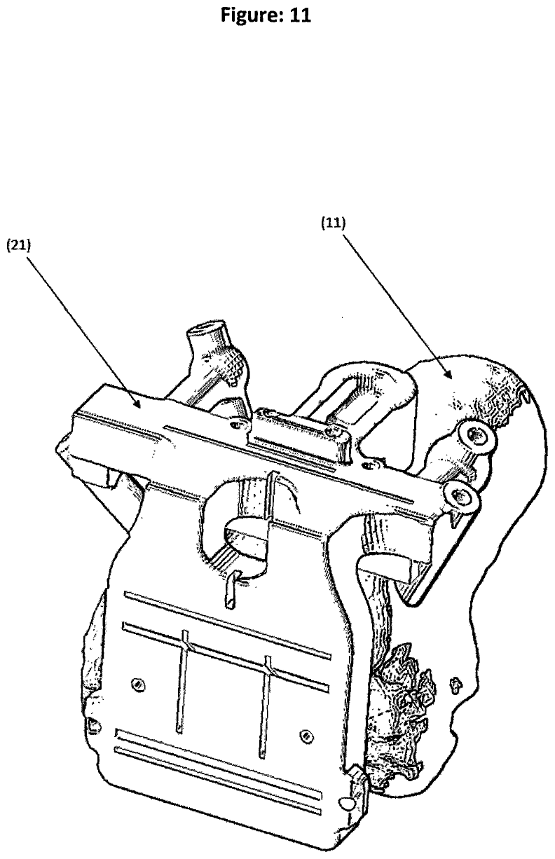

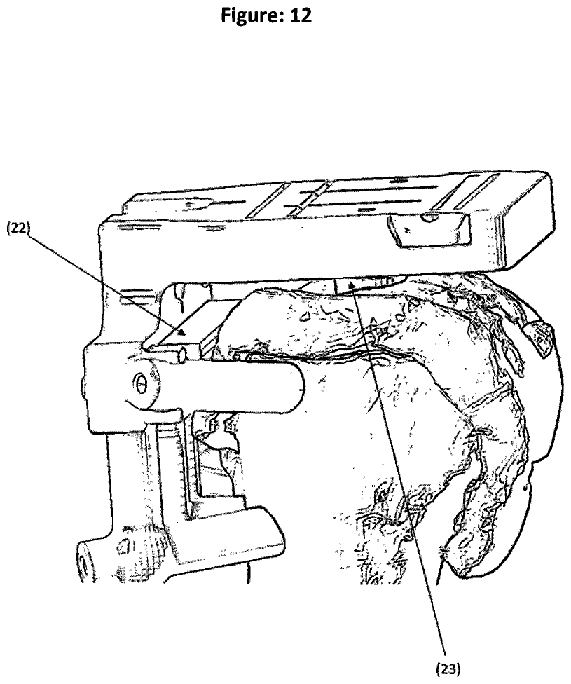

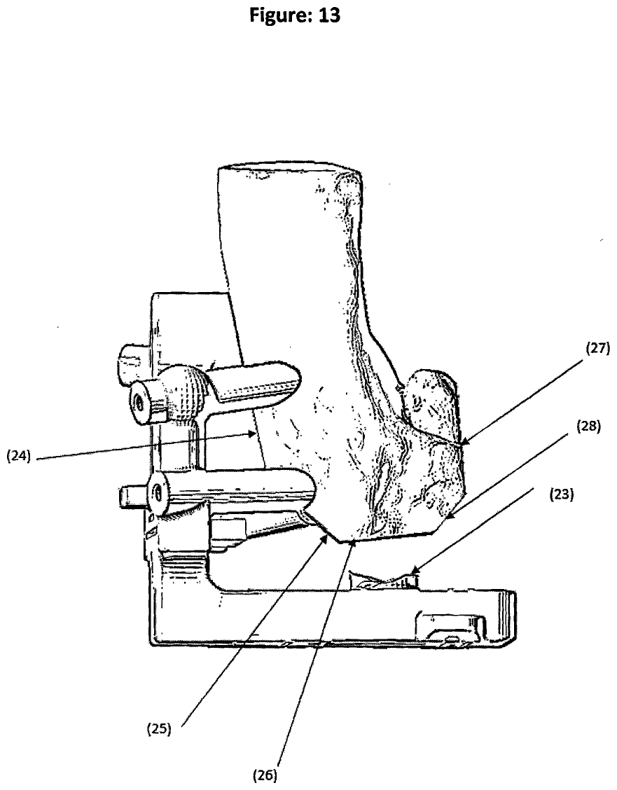

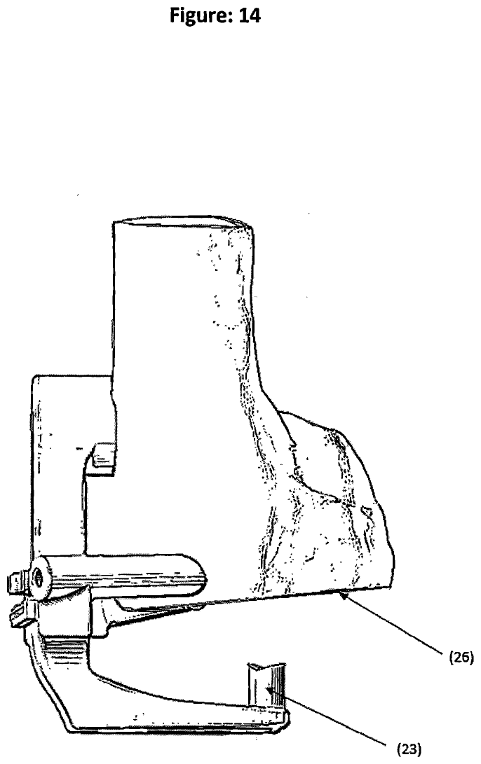

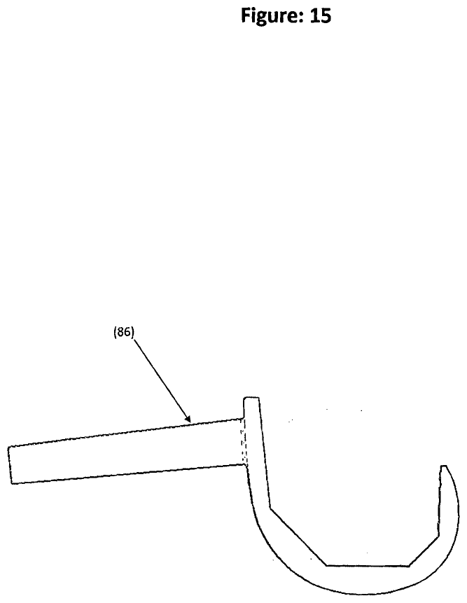

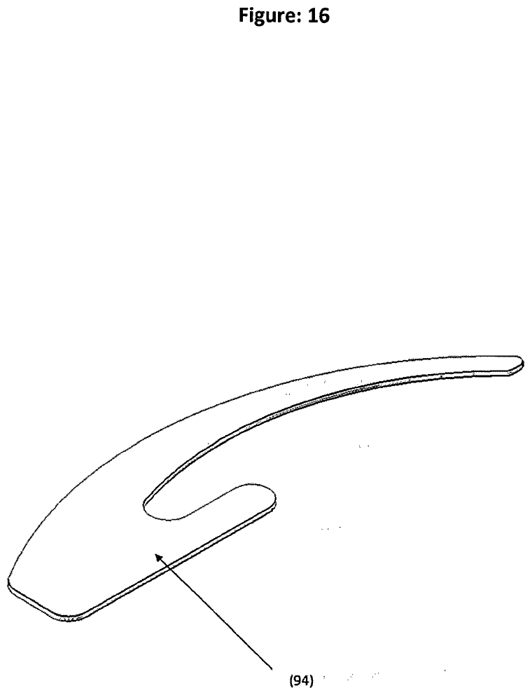

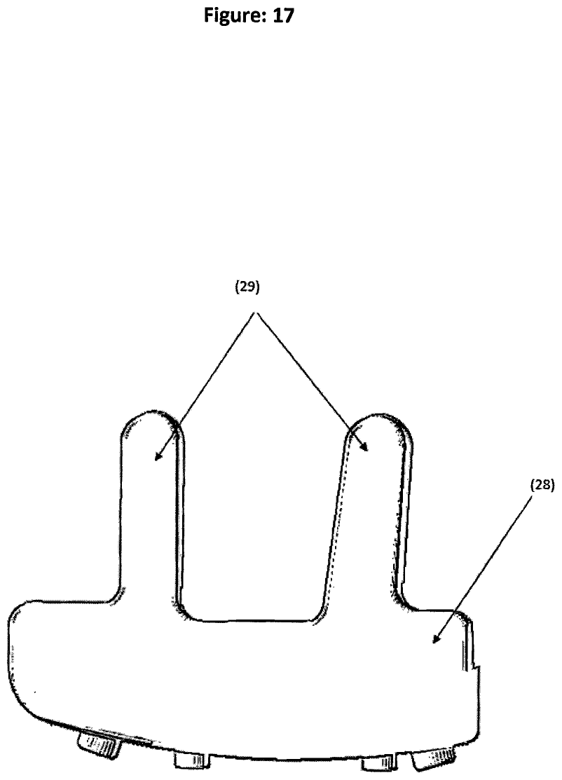

FIG. 17: 3D view of the tibial pin positioning guide (28) shows superior positioning legs (29).

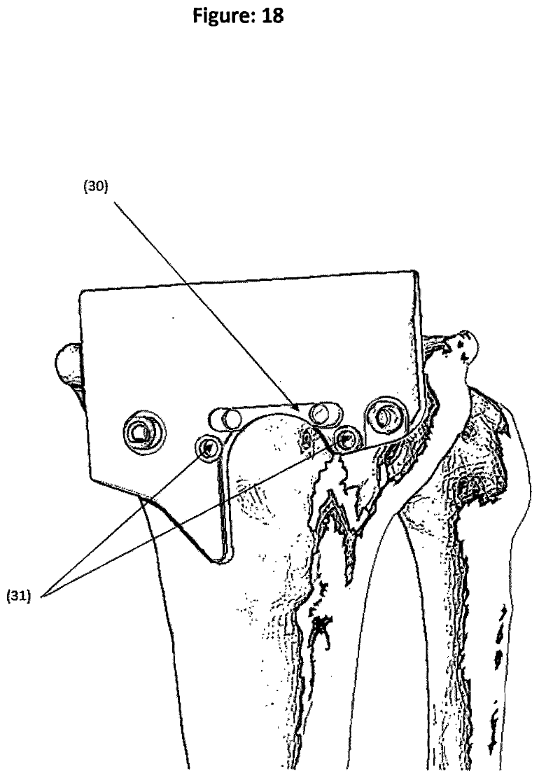

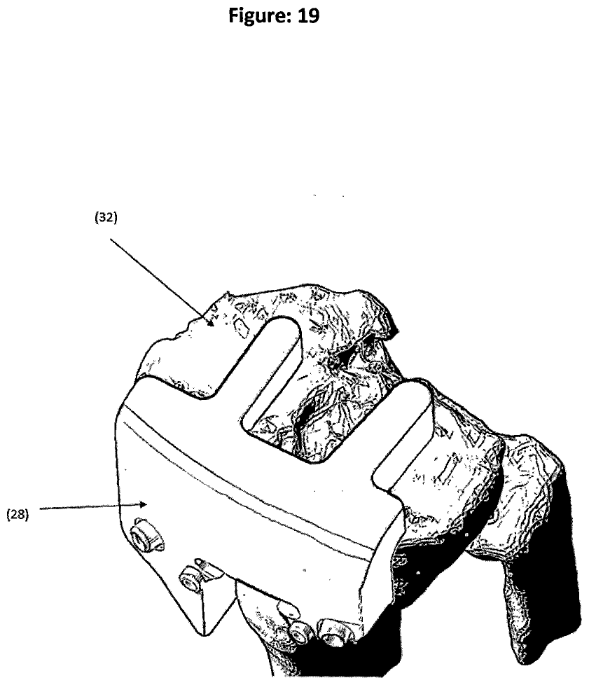

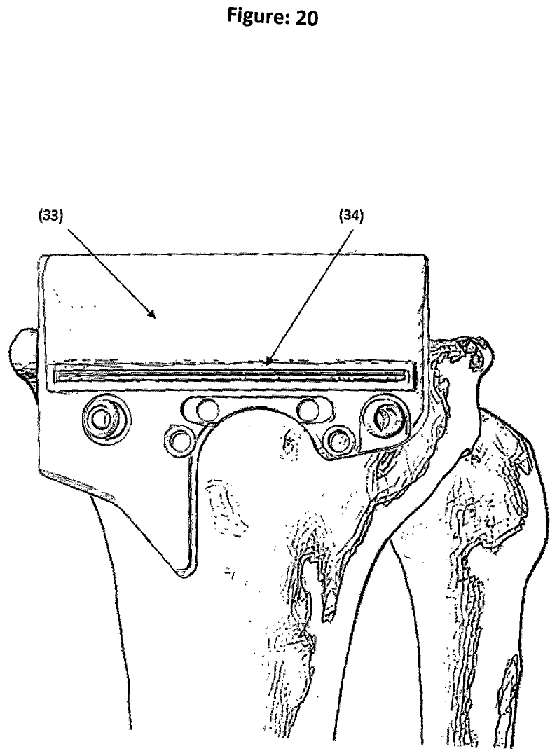

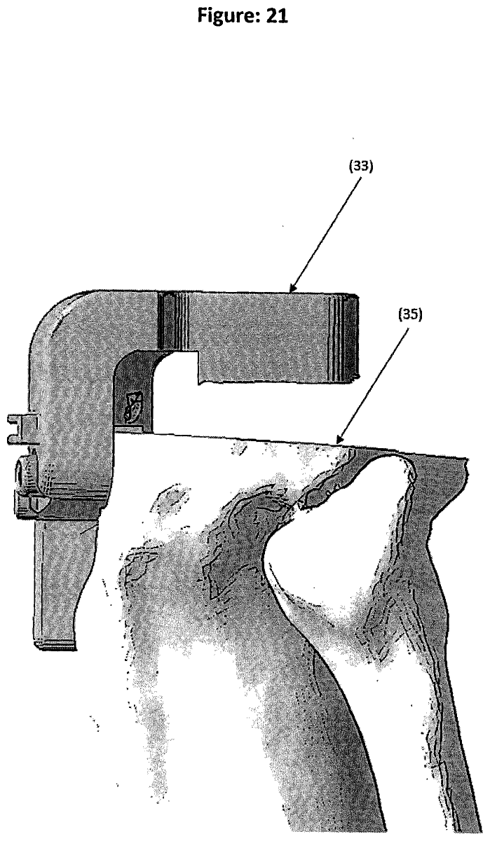

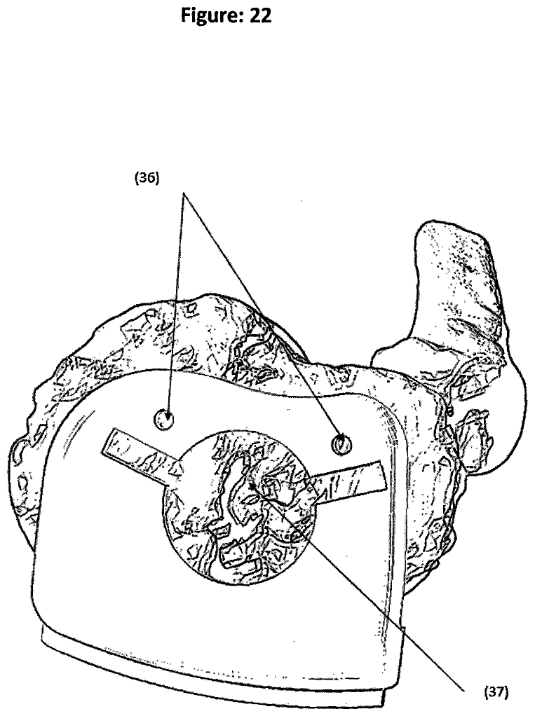

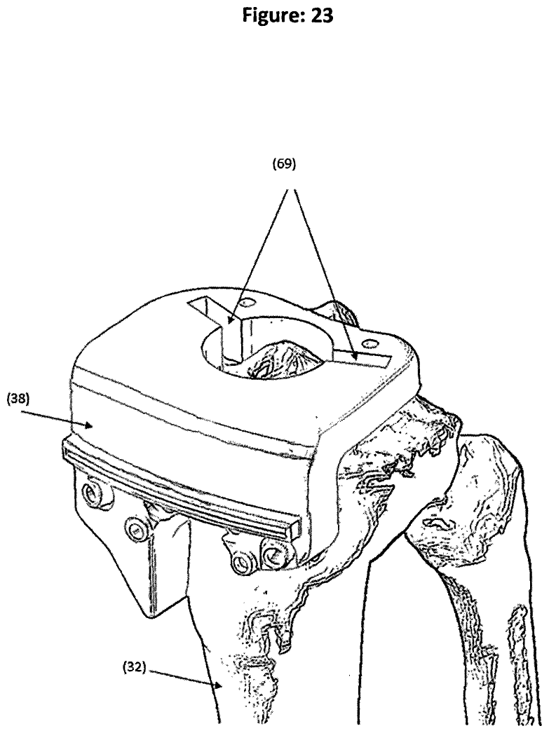

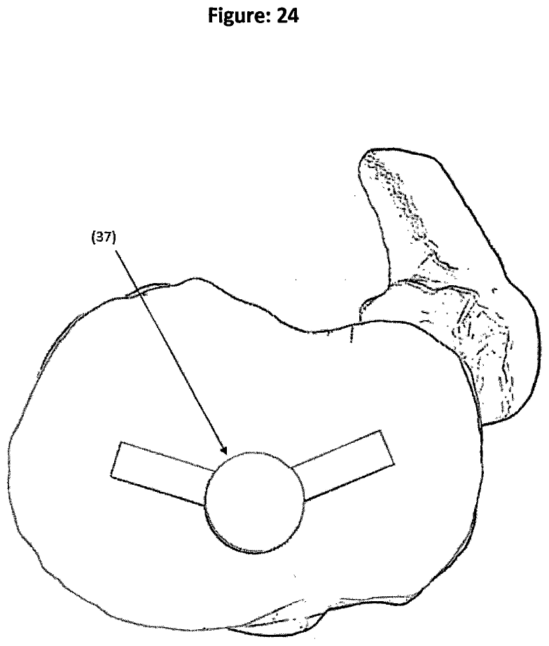

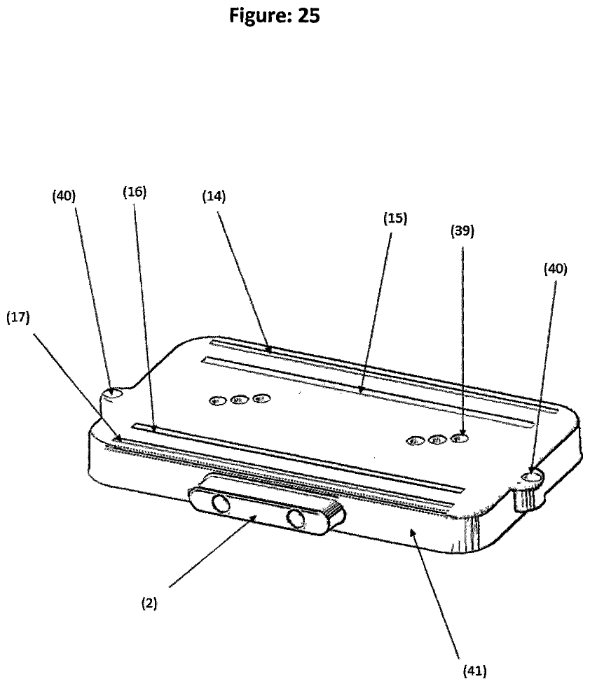

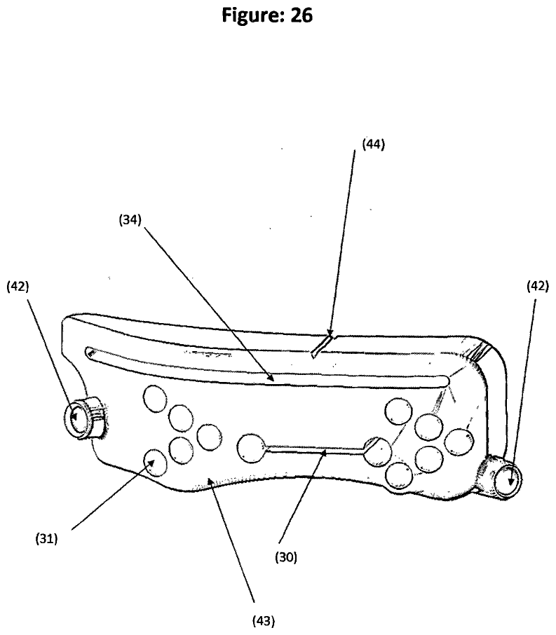

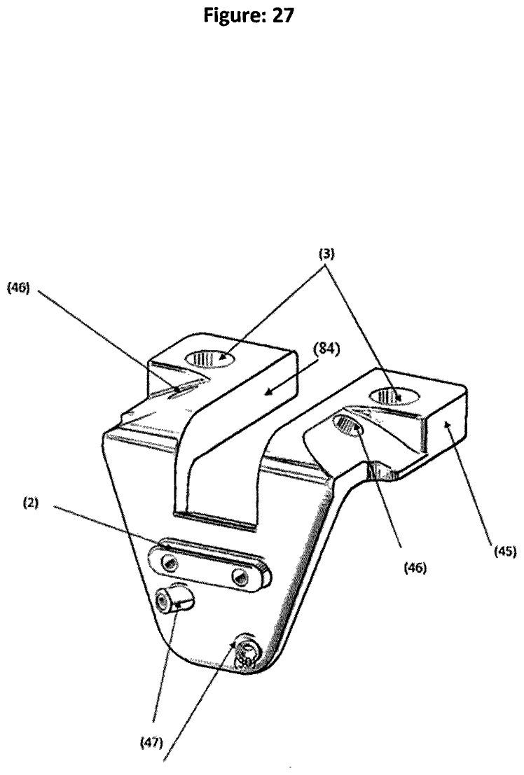

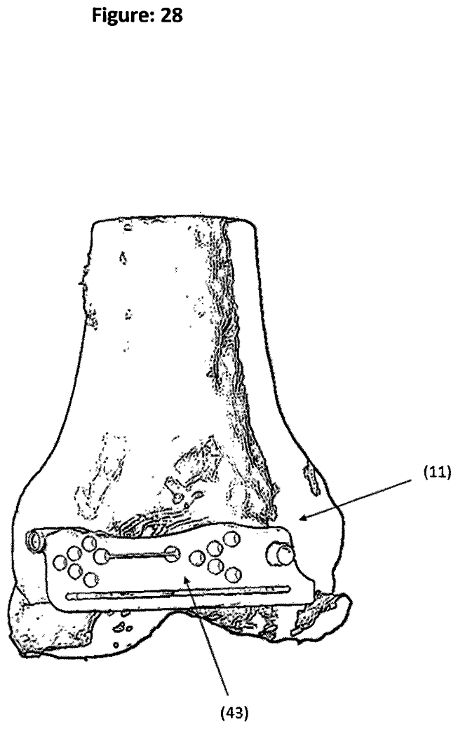

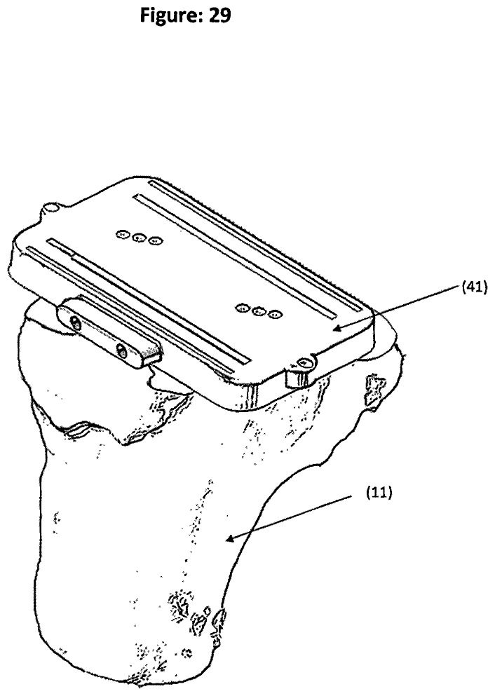

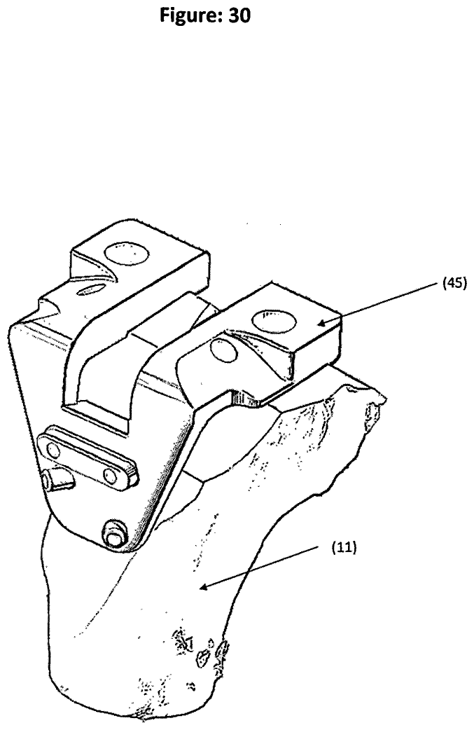

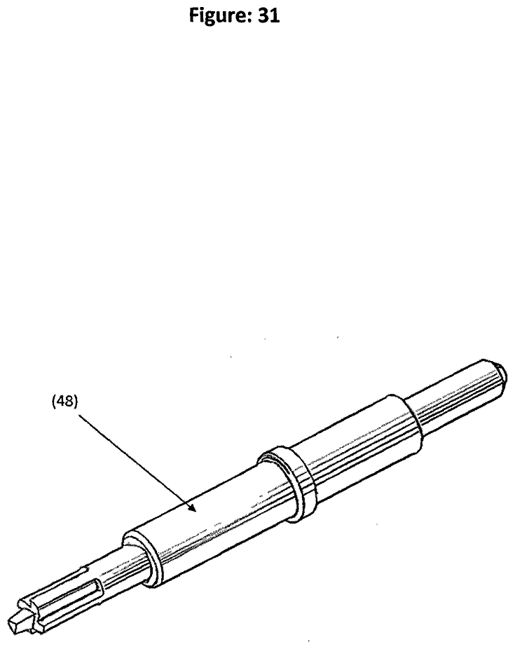

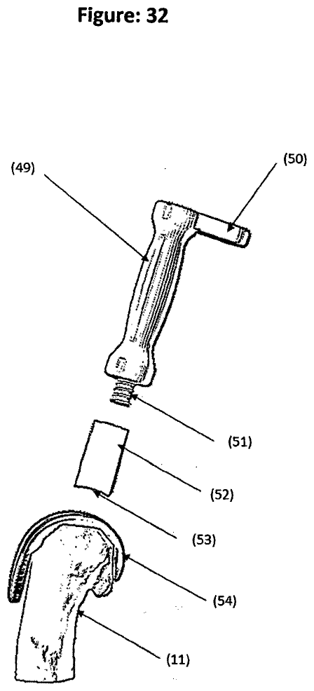

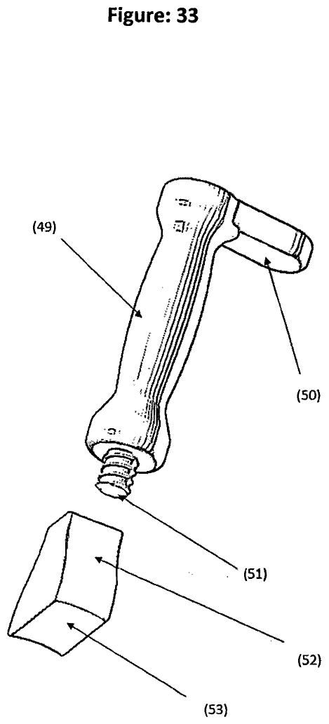

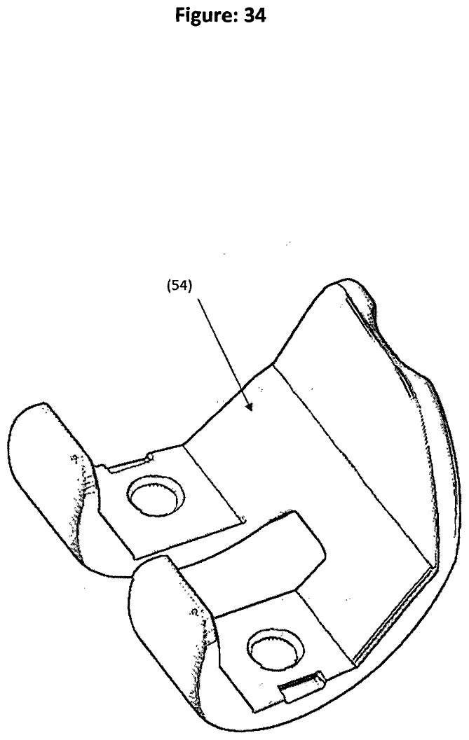

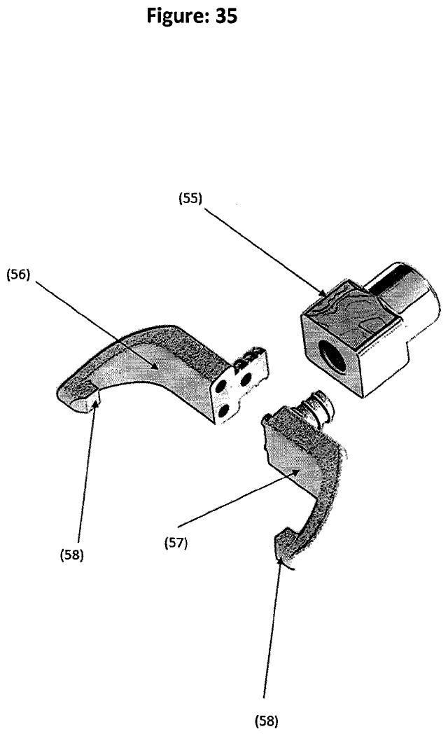

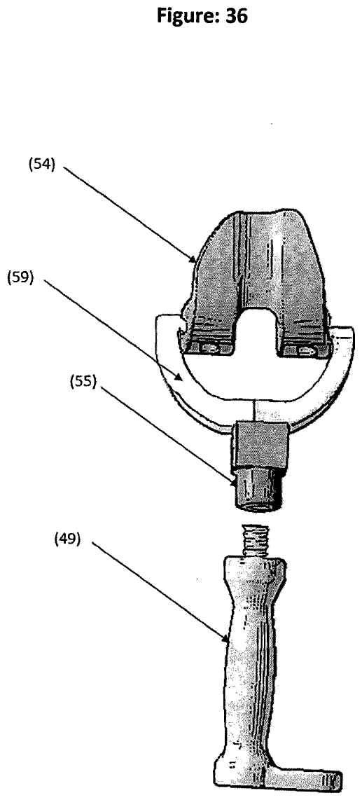

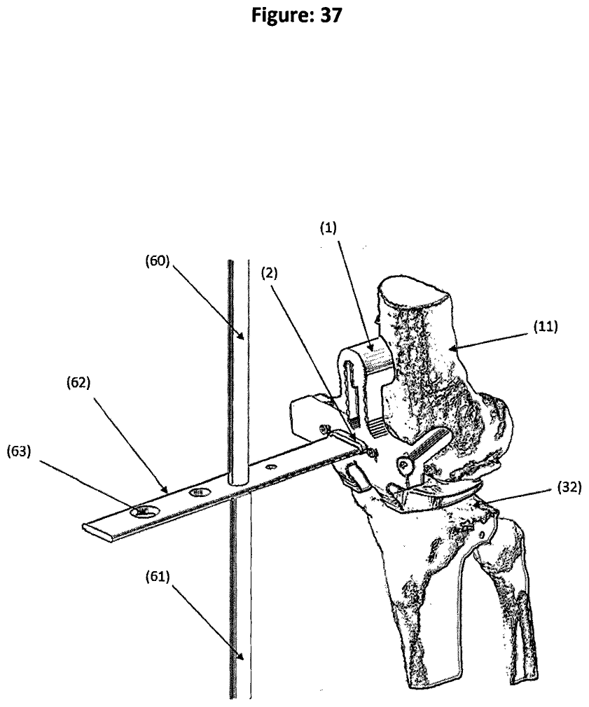

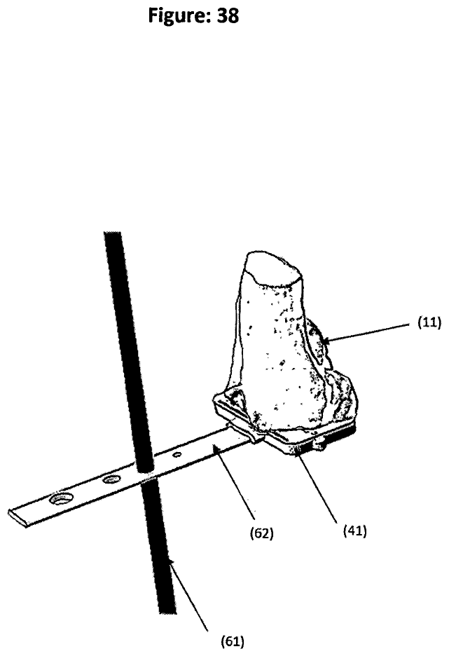

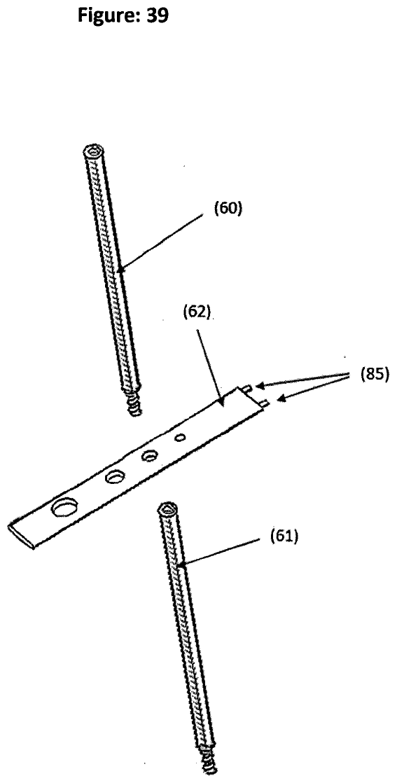

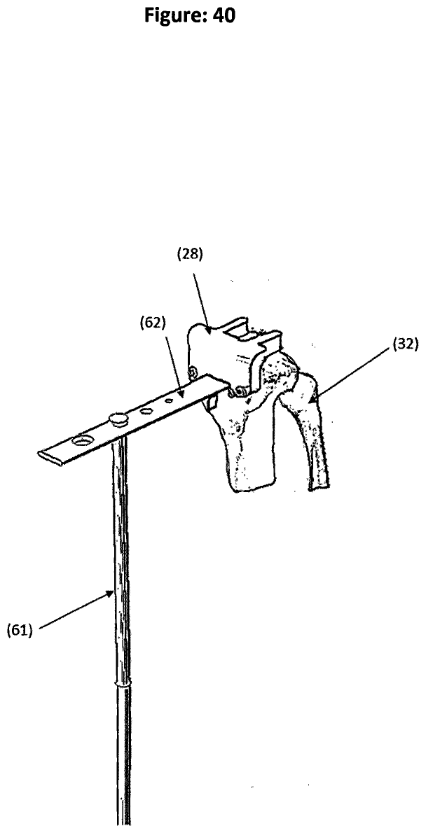

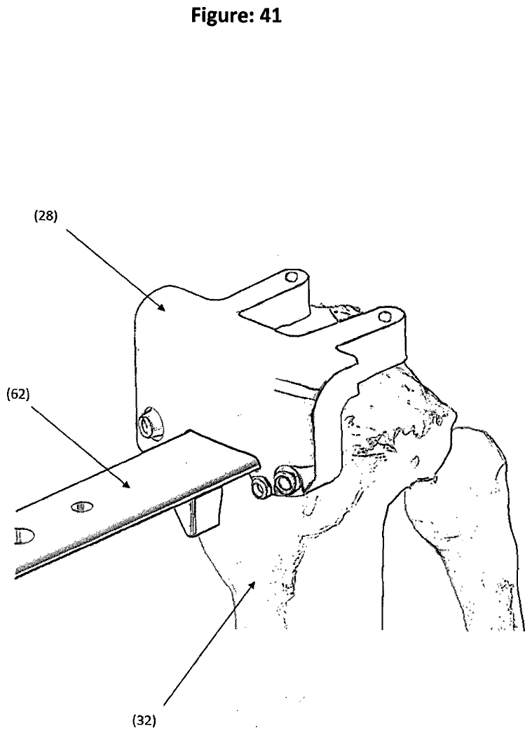

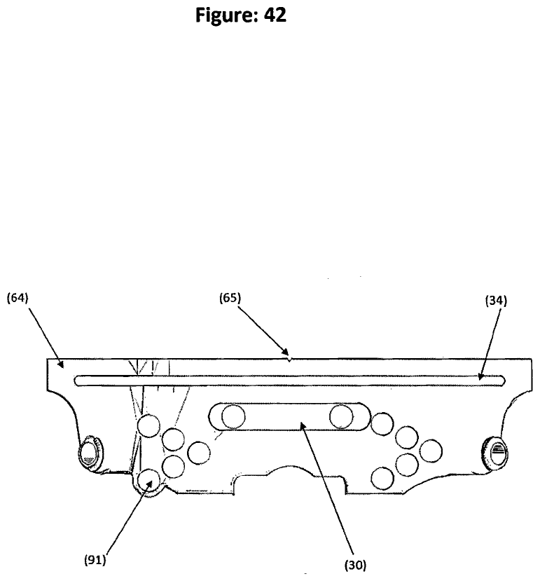

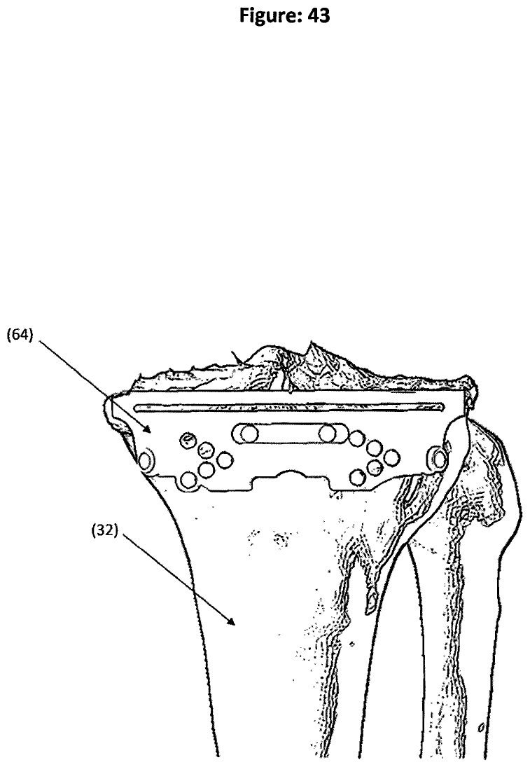

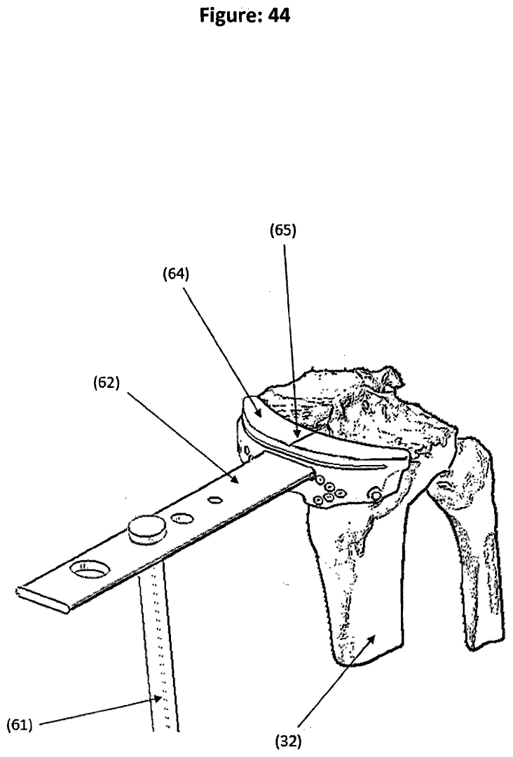

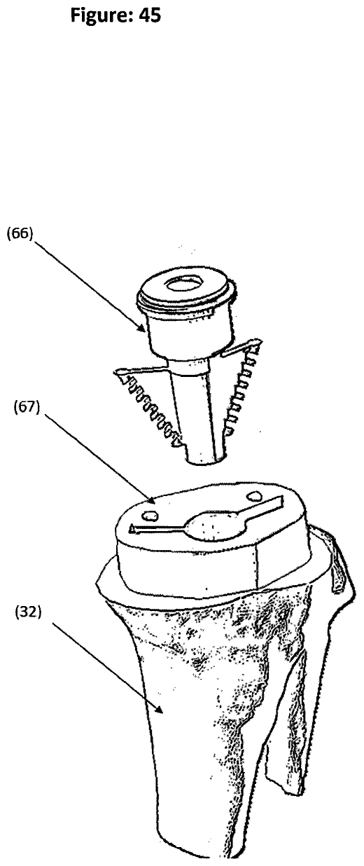

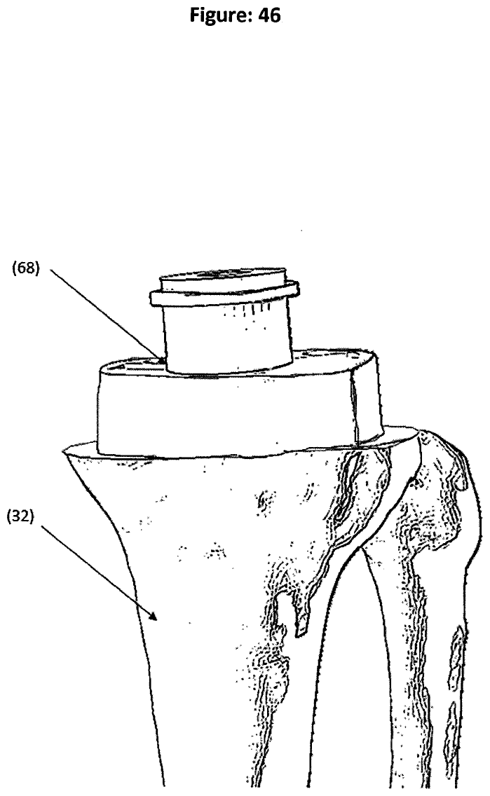

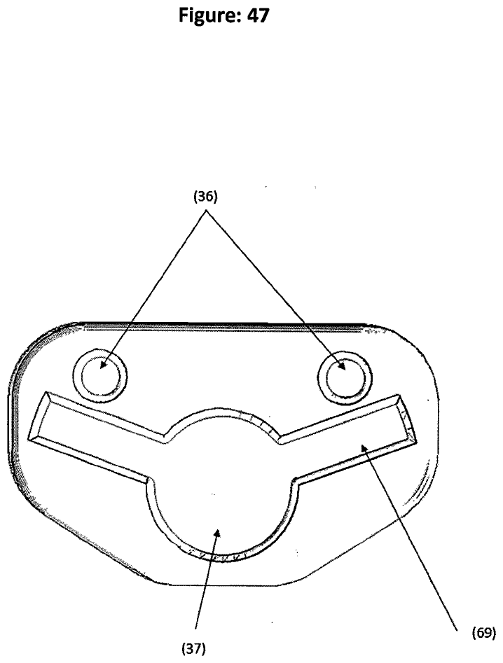

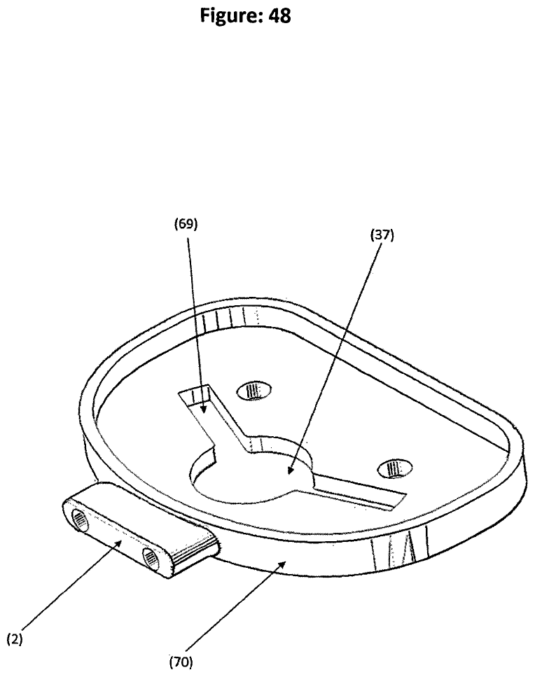

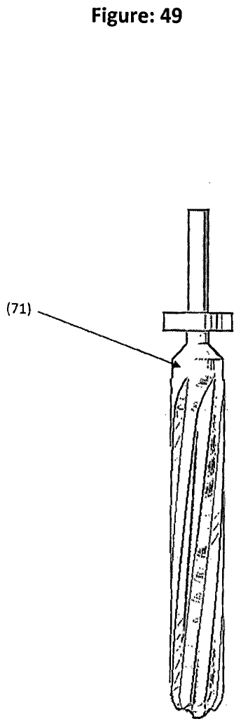

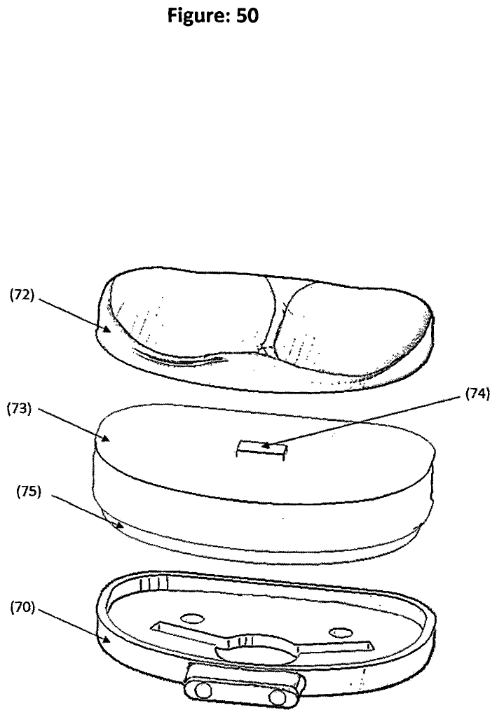

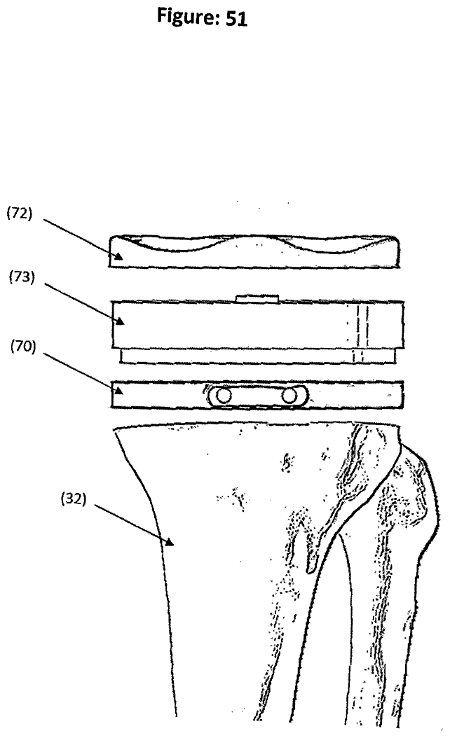

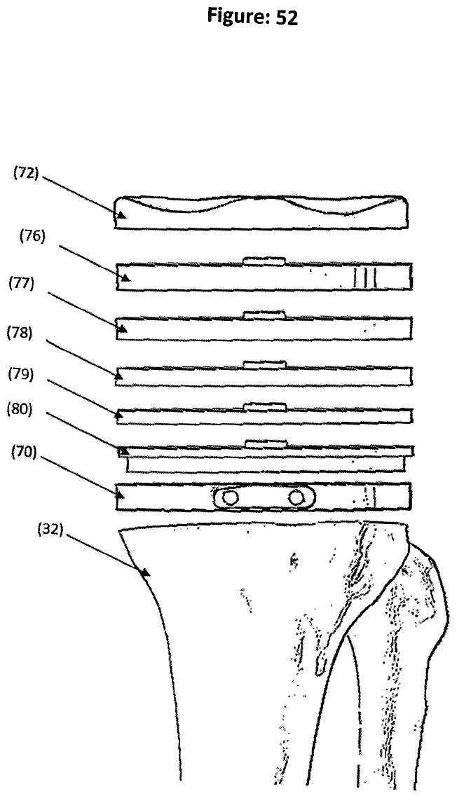

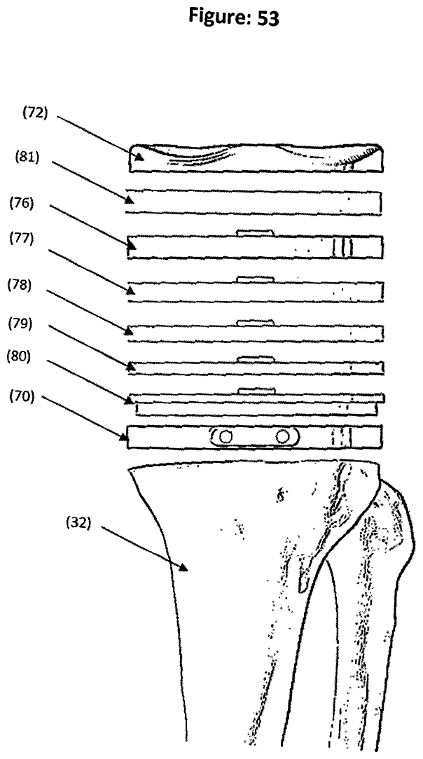

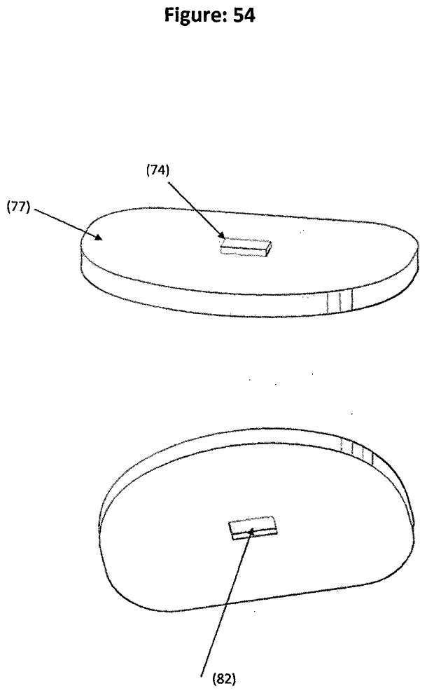

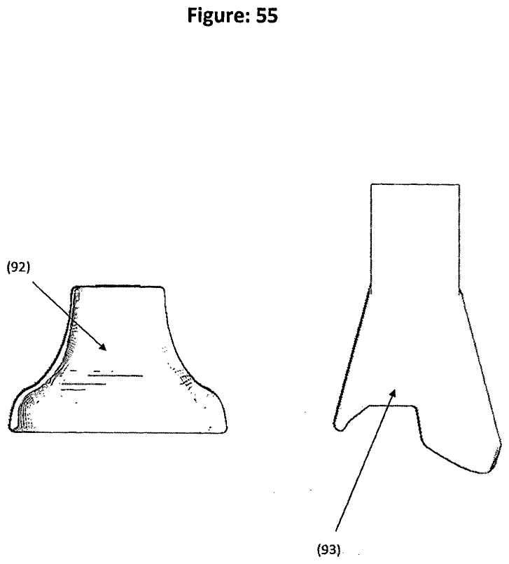

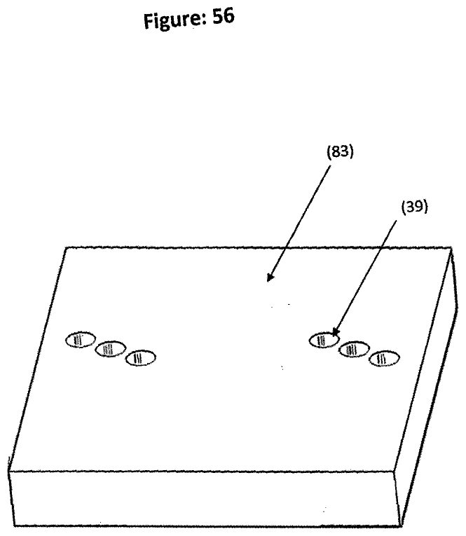

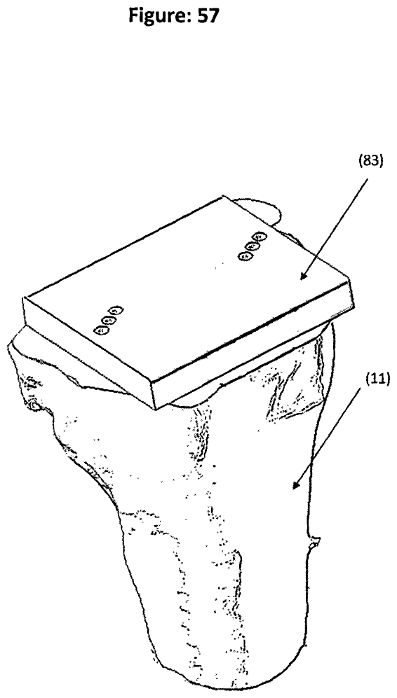

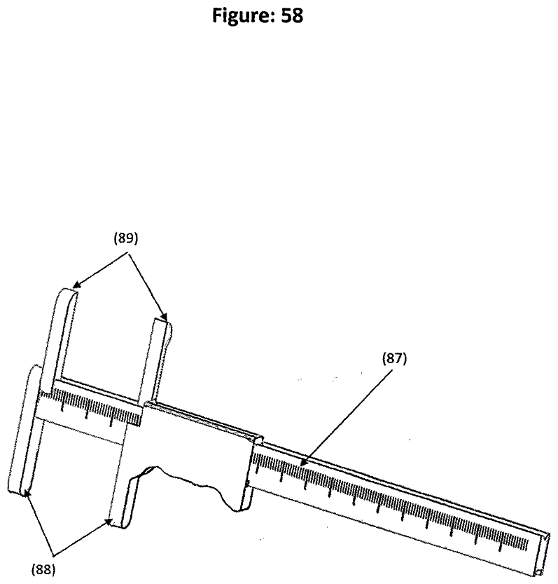

FIG. 18: 3D view of the tibial pin positioning guide shows the proximal tibial component positioning holes (31) and anterior groove for tibial external alignment verification (30).

FIG. 19: 3D view shows the positioning of the tibial pin positing guide (28) over the tibia bone (32).

FIG. 20: 3D view of the tibial proximal cutting guide (33) shows the tibial proximal cutting slot (34).

FIG. 21: 3D view of the tibial proximal cutting guide (33) and the tibia bone after performing the tibial proximal cut (35).

FIG. 22: 3D view of the tibial complete cutting guide shows tibial preparation tower marking holes (36) and the tibia peroration drilling hole (37).

FIG. 23: 3D view shows the positioning of the tibial complete cutting guide (38) over the tibia bone (32).

FIG. 24: 3D view shows tibia bone after performing the keel and stem drilling (37).

FIG. 25: 3D view of the SUI-4in1 femoral cutting block (41) shows the anterior cutting slot (17), the posterior cutting slot (14), the anterior chamfer cutting slot (15), the posterior chamfer cutting slot (16), distal oblique fixation holes (40), alignment tower base (2) and anterior-posterior femoral cut translation holes (39).

FIG. 26: 3D view of the SUI-distal femoral cutting block (43) shows the distal cutting slot (34), anterior oblique fixation holes (42), anterior groove for femoral external alignment verification (30), mediolateral femoral alignment groove (44) and distal-proximal femoral cut translation holes (31).

FIG. 27: 3D view of the SUI-box cutting block (45) shows the box cutting surface (84), alignment tower base (2), distal oblique fixation holes of the SUI-box cutting block (46), anterior oblique fixation holes of the SUI-box cutting block (47) and 4in1 femoral component positioning lugs (3).

FIG. 28: 3D view shows the positioning of SUI-distal femoral cutting block (43) over the femur bone (11).

FIG. 29: 3D view shows the positioning of SUI-4in1 femoral cutting block (41) over the femur bone (11).

FIG. 30: 3D view shows the positioning of SUI-box cutting block (45) over the femur bone (11).

FIG. 31: 3D View of the Lug Holes Drilling Tool (48).

FIG. 32: A schemes of the impaction process shows the impactor/extractor hand (49), impactor/extractor base (50), impactor/extractor screw (51), femoral impactor (52), femoral impaction surface (53), femoral trial (54) and femur bone (11).

FIG. 33: 3D view of impactor/extractor hand (49), impactor/extractor base (50), impactor/extractor screw (51), femoral impactor (52) and femoral impaction surface (53)

FIG. 34: 3D view of femoral trial (54).

FIG. 35: 3D view of the femoral extractor shows femoral extractor head (55), femoral extractor lateral hand (56), femoral extractor medial hand (56) and the femoral extractor hook (58)

FIG. 36: A schemes of the extraction process shows the impactor/extractor hand (49), femoral extractor (59), femoral extractor head (55) and femoral trial (54).

FIG. 37: A schemes of the alignment technique shows the femoral pin positioning guide (1), the femur bone (11), the tibia bone (32), alignment tower base (2), alignment tower plate (62), superior alignment rod portion (60), Inferior alignment rod portion (61) and the positioning holes (63).

FIG. 38: A schemes of the alignment technique shows the SUI-4in1 femoral cutting block (41), the femur bone (11), alignment tower plate (62) and Inferior alignment rod portion (61).

FIG. 39: 3D view of the alignment component shows the alignment tower plate (62), superior alignment rod portion (60), Inferior alignment rod portion (61) and the positioning pins (85).

FIG. 40: A schemes of the alignment technique of the tibia shows the tibial pin positioning guide (28), the tibia bone (32), alignment tower plate (62) and Inferior alignment rod portion (61)

FIG. 41: A schemes of the alignment technique of the tibia shows the tibial pin positioning guide (28), the tibia bone (32) and alignment tower plate (62).

FIG. 42: 2D view of SUI-tibial proximal cutting block (64) shows the anterior groove for tibial external alignment verification (30), tibial proximal cutting slot (34), mediolateral tibial alignment groove (65) and the distal-proximal tibial cut translation holes (91).

FIG. 43: 3D view shows the positioning of SUI-tibial proximal cutting block (64) over the tibia bone (32).

FIG. 44: A schemes of the alignment technique of the tibia shows SUI-tibial proximal cutting block (64), the tibia bone (32), alignment tower plate (62) and Inferior alignment rod portion (61) and mediolateral tibial alignment groove (65).

FIG. 45: 3D view of the tibia preparation technique shows the keel punch (66), the SUI-tibia preparation tower (67) and the tibia bone (32).

FIG. 46: 3D view of the tibia preparation technique shows the keel punch stopper surface (68) and the tibia bone (32).

FIG. 47: 3D view of the tibia preparation tower (67) shows the keel preparation slots (69), tibia preparation tower marking holes (36) and the stem drilling hole (37).

FIG. 48: 3D view of the SUI-trial tibia plateau (70) shows the keel preparation slots (69), alignment tower base (2) and the stem drilling hole (37).

FIG. 49: 3D view of the SUI-tibial preparation drill (71).

FIG. 50: 3D view shows the base surface matching tibia tray (72), thickness utilizing flexion/extension gap shims with 9 mm thickness (73) and SUI-trial tibia plateau (70).

FIG. 51: 3D view of the flexion/extension gap assessment shows the base surface matching tibia tray (72), thickness utilizing flexion/extension gap shims with 9 mm thickness (73), SUI-trial tibia plateau (70) and tibia bone (32).

FIG. 52: 3D view of the flexion/extension gap assessment shows the base surface matching tibia tray (72), thickness utilizing flexion/extension gap shims with different sizes of 5 mm (76), 4 mm (77), 3 mm (78), 2 mm (79), 1 mm (80), SUI-trial tibia plateau (70) and tibia bone (32).

FIG. 53: 3D view of the flexion/extension gap assessment shows the base surface matching tibia tray (72), flexible thickness utilizing flexion/extension gap shims with 3 mm thickness (81), thickness utilizing flexion/extension gap shims with different sizes of 5 mm (76), 4 mm (77), 3 mm (78), 2 mm (79), 1 mm (80), SUI-trial tibia plateau (70) and tibia bone (32).

FIG. 54: 3D view of thickness utilizing flexion/extension gap shims (77) shows the adapting boss (74) and adapting groove (82).

FIG. 55: 3D view of tibial component impact (92) and tibial insert impactor (93).

FIG. 56: 3D view the SUI-balancing block (83) and the anterior-posterior femoral cut translation holes (39).

FIG. 57: 3D view shows the positioning of the SUI-balancing block (83) over the femur bone (11) at the step of flexion balancing.

FIG. 58: 3D view of the caliper (87) shows the gap measuring jaw (89) and thickness measuring jaw (88).

1. A system of patient-matched and surgeon-specific single use surgical instruments designed for five (5) different applications for knee replacement surgeries;
1. Primary total knee replacement
2. Custom-made total knee replacement (primary-revision-hinged)
3. Revision total knee replacement
4. Hinged total knee replacement
5. Uni-compartmental/partial knee replacement
The system comprises patient-matched & surgeon-specific [instruments are designed & prepared for each case based on patient's need and surgeon's demand] set of instruments covering patient-matched set of blocks/guides, “on-demand” surgeon-specific instruments sets, conventional instruments sets, surgical planning software, 3d printing software, 3d printers and sterilization unit.

2. The system as claimed in 1, is designed to plan, develop, produce (via 3D printing technology), package and sterilize single-use surgical instruments for different applications for knee replacement surgeries.

3. the system as claimed in 1, placed inside a healthcare institution or hospital (in-house) or as an independent workshop facility.

4. the system as claimed in 1, delivering knee replacement surgical instruments based on surgeon's demand, according to surgeon's surgical philosophy and approach to surgical philosophy.

5. The system as claimed in 1, is characterized by inclusion or exclusion of a specific surgical instrument(s) is a surgical decision made only by surgeon.

6. The system as claimed in 1, is characterized by producing patient-matched and surgeon-specific single-use instruments based the 2D images of the knee resulting from CT-scans, MRI, Ultrasound and/or X-ray.

7. The system as claimed in 1, is characterized by producing patient-matched and surgeon-specific single-use instruments made from medical-grade and 3D-printable materials that are bioabsorbable, absorbable, non-absorbable, biodegradable, non-biodegradable and recyclable material; wherein all materials in delivered in knee surgical instruments set are biocompatible.

8. The system as claimed in 1, is characterized by producing patient-matched and surgeon-specific single-use instruments made from medical-grade 3D-printable material that is selected from the group of polyamides, polylactic acid and stainless-steel which are successfully utilized for both filament 3D printing and powder 3D printing processes.

9. The system as claimed in 1, producing patient-matched and surgeon-specific single-use instruments for any of the knee implants and fulfils surgical instruments for any surgical applications.

10. The system as claimed in 1, the patient-matched and surgeon-specific blocks/guides, offer three options for the surgeon to choose most appropriate version that suits patient's specific needs and adopted surgical philosophy; each of offered three options consists of femoral component and tibial component.

11. The system as claimed in 1, is characterized by allowing faster and less invasive surgical procedure through skipping the need of intramedullary and/or extramedullary alignment rod designed to validate implant positioning during surgical process.

12. The first option as claimed in 10, is a complete cutting, preparation, and finishing femoral & tibial blocks/guides allowing full surgical access to perform all the joint replacement actions and requirements embedded in bodies of complete cutting, preparation, and finishing femoral & tibial blocks/guides.

13. Femoral component as claimed in 12, consisting of distal femoral cut, anterior femoral cut, posterior femoral cut, anterior chamfer cut, posterior chamfer cut, box cut for PS femoral component, valgus angle, external rotation angle, Q angle, distal femoral component positioning lugs, anterior position verification marking, anterior grooves for external alignment verification, design-specific anterior cut angle, saw blade-specific cutting-slot thickness and oblique fixation holes.

14. The tibial component as claimed in 12, consisting of proximal tibia cut, proximal tibia cut posterior slope angle, tibia preparation drill(s), tibia preparation keel/punch, anterior position verification marking, anterior grooves for external alignment verification, saw blade-specific slot thickness and oblique fixation holes.

15. The second option as claimed in 10, is an initial cutting blocks/guides offering guides for preparation and finishing of femoral and tibial aspects with features embedded in bodies of initial cutting components.

16. Femoral component as claimed in 15, consisting of distal femoral cut, valgus angle, external rotation angle, q angle, 4in1 cutting block marking, distal femoral component positioning lugs marking, anterior position verification marking, anterior grooves for external alignment verification, saw blade-specific cutting-slot thickness and oblique fixation holes.

17. The tibial component as claimed in 15, consisting of proximal tibia cut, proximal tibia cut posterior slope angle, anterior position verification marking, anterior grooves for external alignment verification, saw blade-specific slot thickness and oblique fixation holes.

18. The third option as claimed in 10, is a pin-positioning blocks/guides offering guides for cutting, preparation and finishing of femoral and tibial aspects with features embedded in bodies of pin-positioning blocks/guides.

19. Femoral component as claimed in 18, consisting of distal femoral cut marking, valgus angle, external rotation angle, Q angle, 4in1 cutting block marking, distal femoral component positioning lugs marking, anterior position verification marking, anterior grooves for external alignment verification, saw blade-specific cutting-slot thickness and oblique fixation holes.

20. The tibial component as claimed in 18, consisting of proximal tibia cut marking, proximal tibia cut posterior slope angle, anterior position verification marking, anterior grooves for external alignment verification, saw blade-specific slot thickness and oblique fixation holes.

21. The guide as claimed in 10, is characterized by a marking central slot in allows validation of guide position, alignment, and rotation over the bone throughout surgical procedure.

22. The guide as claimed in 10, is characterized by alignment slots allow the surgeon to fix a multi-sizes alignment plate and rod to ensure the implant alignment along varus/vulgus, internal/external rotation and axial rotation throughout surgical procedure.

23. The system as claimed in 1, delivers single-use patient-matched and surgeon-specific conventional instruments “based on surgeon's demand” with different eleven options.

24. Option 1 as claimed in 23, is designed for primary total knee replacement, custom-made total knee replacement (primary-revision-hinged), revision total knee replacement, hinged total knee replacement and uni-compartmental/partial knee replacement; consisting of distal femoral cutting block offers open or sloped cut option, minimally invasive or component dimension option, distal-proximal femoral cut translation [on demand (0 mm), (−1/+1 mm), (−/+2 mm) (−/+3 mm) and (−/+4 mm)] options, anterior grooves for external alignment verification, position validation central mark, saw blade-specific slot thickness, and oblique fixation holes.

25. Option 2 as claimed in 23, is designed for primary total knee replacement, custom-made total knee replacement (primary-revision-hinged), revision total knee replacement and hinged total knee replacement; consisting of distal femoral cutting block offers open or sloped cut option, minimally invasive or component dimension option, distal-proximal femoral cut translation [on demand 0 mm up-to 40 mm in 4 mm increment] options, anterior grooves for external alignment verification, position validation central mark, saw blade-specific slot thickness, and oblique fixation holes.

26. Option 3 as claimed in 23, is designed for primary total knee replacement, custom-made total knee replacement (primary-revision-hinged), revision total knee replacement and hinged total knee replacement; consisting of 4in1 femoral cutting block offers open or sloped cut option, minimally invasive or component dimension option, anterior-posterior femoral cut translation [on demand (0 mm), (−1/+1 mm), (−/+1.5 mm) (−/+2 mm) and (−/+3 mm)] options, anterior grooves for external alignment verification, position validation central mark, saw blade-specific slot thickness, and oblique fixation holes.

27. Option 4 as claimed in 23, is designed for primary total knee replacement, custom-made total knee replacement (primary-revision-hinged), revision total knee replacement and hinged total knee replacement; consisting of cutting block for box cut for ps femoral component offers open or sloped cut option, minimally invasive or component dimension option, distal femoral component positioning lugs, anterior grooves for external alignment verification, position validation central mark, saw blade-specific slot thickness, and oblique fixation holes.

28. Option 5 as claimed in 23, is designed for uni-compartmental/partial knee replacement; consisting of 3in1 femoral cutting block offers open or sloped cut option, minimally invasive or component dimension option, anterior grooves for external alignment verification, saw blade-specific slot thickness, and oblique fixation holes.

29. Option 6 as claimed in 23, is designed for primary total knee replacement, custom-made total knee replacement (primary-revision-hinged), revision total knee replacement, hinged total knee replacement and uni-compartmental/partial knee replacement; consisting of proximal tibia cutting block offers open or sloped cut option, minimally invasive or component dimension option, distal-proximal tibial cut translation [on demand (0 mm), (−1/+1 mm), (−/+2 mm) (−/+3 mm) and (−/+4 mm)] options, anterior grooves for external alignment verification, position validation central mark, saw blade-specific slot thickness, and oblique fixation holes.

30. Option 7 as claimed in 23, is designed for primary total knee replacement, custom-made total knee replacement (primary-revision-hinged), revision total knee replacement and hinged total knee replacement; consisting of proximal tibia cutting block offers open or sloped cut option, minimally invasive or component dimension option, distal-proximal tibial cut translation [on demand 0 mm up-to 40 mm in 4 mm increment] options, anterior grooves for external alignment verification, position validation central mark, saw blade-specific slot thickness, and oblique fixation holes.

31. Option 8 as claimed in 23, is designed for primary total knee replacement, custom-made total knee replacement (primary-revision-hinged), revision total knee replacement, hinged total knee replacement and uni-compartmental/partial knee replacement; consisting of trial instruments set offers patient size-specific femoral trial, tibia plate trial, tibia insert trials [surface & multiple shims], flexion/extension gap assessment shims, intra-medullary stem extensions [0/offset tibial & femoral stems], intra-medullary sleeves [tibial & femoral sleeves], intra-medullary cones [tibial & femoral cones], tibia wedges [full, hemi, step & block wedges] and femoral augments [distal, anterior & posterior].

32. Option 9 as claimed in 23, is designed for primary total knee replacement, custom-made total knee replacement (primary-revision-hinged), revision total knee replacement, hinged total knee replacement and uni-compartmental/partial knee replacement; consisting of position verification & validation instruments set, allows intra-operative validation to make a double check before performing the final bone resection, offers full limb external alignment plate & rods [plate dimensions, rod diameter & rod length are on surgeon's demand], femoral cut validation c-section tool, tibia bone cut stylus, tibial & femoral bone cuts verification wing, and bone caliber.

33. Option 10 as claimed in 23, is designed for primary total knee replacement, custom-made total knee replacement (primary-revision-hinged), revision total knee replacement, hinged total knee replacement and uni-compartmental/partial knee replacement; consisting of bone preparation and finishing set offers femoral lugs drill, tibia preparation drill, tibia keel/punch preparation, sleeves preparation, stem extension preparation, long-stem preparations, cones preparation and other preparation and finishing instruments based on surgeon's demand

34. Option 11 as claimed in 23, is designed for primary total knee replacement, custom-made total knee replacement (primary-revision-hinged), revision total knee replacement, hinged total knee replacement and uni-compartmental/partial knee replacement; consisting of impaction & extraction instruments set offers modular impaction/extraction handle, femoral trial & final femoral component positioning impactor/extractor, femoral impactor, tibia base plate impactor, tibia insert impactor.

35. The system as claimed in 23, is characterized by a balancing block for the purpose of flexion/extension gap balancing.

36. The balancing block as claimed in 35, contains translation holes with +1, −1, +2, −2 to ensure balancing anterior up of posterior down.

37. The system as claimed in 1, delivers single-use patient-matched and surgeon-specific blocks/guides and conventional instruments that exhibit minimal scratching and no observable debris generation during use.

38. The system as claimed in 1, comprises one or more material suitable for maintaining sterility of knee replacement surgical instrument contained therein during transportation or storage thereof.

39. The system as claimed in 1, wherein the one or more materials are selected from the group consisting of stainless-steel, high-density polyamide, polylactic acid, polyethylene, polycarbonate, acrylonitrile-butadiene-styrene, laminates of one or more of these materials, coextruded filament including one or more of these materials, and combinations thereof.

40. The system as claimed in 1, delivers single-use knee replacement sets of instruments each of these sets is designed for a specific patient based on the surgical needs of such specific patient and surgical philosophy of operating surgeon.

41. The system as claimed in 1, operates within specifications of “clean room” to minimize rates of contamination and pollutants such as dust, airborne microbes and aerosol particles during the manufacturing and sterilization process.
